(19) United States
(12) Patent Application Publication
(10) Pub. No.: US 2025/0381002 A1
(43) Pub. Date: Dec. 18, 2025

(54) BELT-TYPE REMOTE CENTER OF MOTION MECHANISM AND ROBOT FOR MINIMALLY INVASIVE SURGERY EQUIPPED WITH THIS MECHANISM

(71) Applicant: KOREA INSTITUTE OF SCIENCE AND TECHNOLOGY, Seoul (KR)

(72) Inventors: Chunwoo KIM, Seoul (KR); Dong Eun CHOI, Seoul (KR)

(73) Assignee: KOREA INSTITUTE OF SCIENCE AND TECHNOLOGY, Seoul (KR)

(21) Appl. No.: 19/222,486

(22) Filed: May 29, 2025

(30) Foreign Application Priority Data
Jun. 12, 2024 (KR) 10-2024-0076530

(51) Int. Cl. A61B 34/00; A61B 34/30; B25J 9/10

(52) U.S. Cl. CPC A61B 34/71; A61B 34/72; B25J 9/104; A61B 34/30; A61B 2034/302

(57) ABSTRACT

A belt-type remote center of motion mechanism according to an embodiment of the present disclosure, comprising: an input link; a first pulley positioned toward a first end side of the input link and a second pulley positioned spaced apart from the first pulley; a power transmission member including a belt transmitting a rotation of the first pulley to the second pulley; a first reducer located between the first pulley and the first end side of the input link and configured to reduce a rotational speed of the first pulley and transmit it to the input link; an output link having a first end coupled to the second pulley and receiving a rotation of the second pulley; and a second reducer positioned between the second pulley and the first end of the output link and configured to reduce a rotational speed of the second pulley and transmit it to the output link.

### CROSS-REFERENCE TO RELATED APPLICATION

This application claims priority to and the benefit of Korean Patent Application No. 10-2024-0076530 filed in the Korean Intellectual Property Office on Jun. 12, 2024.

### BACKGROUND OF THE INVENTION

### (a) Field of the Invention

The present disclosure relates to a belt-type remote center of motion mechanism and a robot for minimally invasive surgery having the same.

### (b) Description of the Related Art

Minimally invasive surgery is a surgical method that uses fewer incisions to minimize scarring on the body.

An example of minimally invasive surgery is laparoscopic surgery, which is performed by making several small 0.5 to 1.5 cm holes (i.e., incisions) in the abdomen through which a video camera and other instruments are inserted and maneuvered, as opposed to open surgery, which is traditionally performed through a large incision in the abdomen.

Robotic surgery also falls under the category of minimally invasive surgery. Robotic surgery is based on the same principle as laparoscopic surgery, but differs in that the surgeon controls a robotic arm to perform the surgery, more freedom of movement and fine, jitter-free surgery.

Minimally invasive surgery involves less damage to the area, resulting in less pain and pulmonary complications, and faster recovery and return to normal life.

As illustrated in FIG. 1, a surgical tool of the robot used for minimally invasive surgery performs four degrees of freedom motion: two rotations, a Tilt (i.e., pitch) motion and a Pan (i.e., roll) motion, centered on the human body entry point (i.e., fulcrum point), an insertion motion of the surgical tool, and an axial rotation (i.e., spin) motion of the surgical tool.

The movements of these surgical tools can be implemented by controlling the movements of a general 6-DOF (Degrees Of Freedom) robot, but due to safety issues in the event of control failure, most minimally invasive surgical robots use a mechanism that mechanically implements 4-DOF movements.

In a remote center of motion (i.e., RCM) mechanism, various methods have been proposed to implement two-degree-of-freedom rotation of a surgical tool around a fixed point (i.e., RCM point) outside the mechanism.

Traditional remote center of motion mechanisms include isocenter type, circular tracking arc type, parallelogram type, belt-type, spherical linkage type, gimbal type, parallel wrist type, and gear train type.

Among these, a belt-type remote center of motion mechanism is configured to implement a remote center of motion by constraining rotation between the links, such as through a belt or rope.

To illustrate shown in FIG. 2, a pulley P1′ is coupled to one end side of an input link IL and a pulley P2′ is coupled to the other end side of the input link IL, and a fixed pulley P1′ and a rotary pulley P2′ are connected by a belt B.

The fixed pulley P1′ rotates around an axis A1′ while its position is fixed by the axis A1′. When the input link IL rotates around the fixed pulley P1′, the rotating pulley P2′ revolves around the fixed pulley P1′ together with the input link IL and rotates around the axis A2′ by the belt B.

Thus, when the input link IL is rotated counterclockwise θ with respect to the fixed pulley P1′, the belt B is rotated by −θ, and the output link OL remains horizontal, thus implementing a remote center of motion mechanism.

Here, rotating the belt B by −θ means rotating it θ in the opposite direction to the direction of rotation of the input link IL, e.g., clockwise in FIG. 2. Here, the belt B rotating by −θ means rotating by θ in the opposite direction to the rotational direction of the input link IL, for example, clockwise in FIG. 2.

Therefore, belt-type remote center of motion mechanisms are widely applied in existing commercial surgical robots because they can achieve remote center of motion while using fewer links.

However, in a traditional belt-type remote center of motion mechanism, the link and pulley are directly connected, so the torque acting on the link is transferred directly to the tension in the belt.

Therefore, as illustrated in FIG. 3, displacement of the surgical robot occurs as the belt B stretches. That is, a displacement of Δh occurs in the output link OL of the surgical robot.

Therefore, to increase the stiffness of the surgical robot, the diameter of the pulleys P1′ and P2′ must be increased or the stiffness of the belt B must be increased.

However, the timing belt that constitutes the belt B is made of elastic material, so there is a limit to increase the stiffness, and if a wire or rope with higher stiffness than the timing belt B is used, it is difficult to assemble and adjust the tension, and the slippage of the wire or rope reduces the accuracy of the operation of the output link OL.

Furthermore, in the case of a conventional belt-type remote center of motion mechanism, an actuator for the insert and exit motion of the surgical tool and an actuator for the axial rotation motion of said surgical tool are mounted on the output link OL.

And a relatively large actuator is needed to move the surgical tool in and out.

Therefore, there is a problem that the installation of the above actuators increases the load and inertia of the robot.

### SUMMARY OF THE INVENTION

The present disclosure is to solve at least one of the above-described problems.

Another technical object of the present disclosure is to provide a belt-type remote center of motion mechanism and a minimally invasive surgical robot equipped with the same, which can be utilized for minimally invasive surgery on hard tissues (bones, joints, etc.) requiring great force.

Another technical object of the present disclosure is to provide a belt-type remote center of motion mechanism and a minimally invasive surgical robot equipped with the same, which has enhanced rigidity by reducing a rotational speed of a pulley by a reducer and then transmitting it to a link to reduce the tension transmitted to a belt.

Another technical object of the present disclosure is to provide a belt-type remote center of motion mechanism and a minimally invasive surgical robot equipped with the same, which maintains constraints for remote center of motion by adjusting the gear ratio of a reducer.

Another technical object of the present disclosure is to provide a belt-type remote center of motion mechanism and a minimally invasive surgical robot equipped with the same, which can drive not only 2-DOF rotation (pan/tilt) around a remote center of motion (i.e., human body entry point) but also insertion motion of a surgical tool, by an actuator located at the base of the robot.

Another technical object of the present disclosure is to provide a belt-type remote center of motion mechanism and a minimally invasive surgical robot equipped with the same, which can reduce the mass and size of the output link portion.

Another technical object of the present disclosure is to provide a belt-type remote center of motion mechanism and a minimally invasive surgical robot equipped with the same, which can reduce interference between output links when using multiple surgical robotic arms.

Another technical object of the present disclosure is to provide a belt-type remote center of motion mechanism and a minimally invasive surgical robot equipped with the same, which maintains constraints for remote center of motion even during the insertion/exit motion of a surgical tool.

The technical objects to be achieved by the present disclosure are not limited to those that have been described hereinabove merely by way of example, and other technical objects that are not mentioned can be clearly understood by those skilled in the art, to which the present disclosure pertains, from the following descriptions.

A belt-type remote center of motion mechanism according to an embodiment of the present disclosure, comprising: an input link; a first pulley positioned toward a first end side of the input link and a second pulley positioned spaced apart from the first pulley; a power transmission member including a belt transmitting a rotation of the first pulley to the second pulley; a first reducer located between the first pulley and the first end side of the input link and configured to reduce a rotational speed of the first pulley and transmit it to the input link; an output link having a first end coupled to the second pulley and receiving a rotation of the second pulley; and a second reducer positioned between the second pulley and the first end of the output link and configured to reduce a rotational speed of the second pulley and transmit it to the output link.

The first reducer can reduce the rotational speed of the first pulley with a gear ratio of N₁:1 and transmit it to the input link, and the second reducer can reduce the rotational speed of the second pulley with a gear ratio of N₂:1 and transmit it to the output link, where N₂ can be −(N₁−1).

When the rotation angle of the first pulley is θ, a rotation angle α1 of the first link may be θ/N₁, and a rotation angle α2 of the second link may be −θ/N₁.

The first reducer and the second reducer may include a harmonic drive or a planetary gear assembly.

For example, the first reducer may include a first harmonic drive, and the second reducer may include a second harmonic drive.

In this case, the first harmonic drive may include a wave generator acting as an input, a circular spline acting as an output, and a fixed flex spline.

And the second harmonic drive may include a wave generator acting as an input, a flex spline acting as an output, and a fixed circular spline.

In this way, the gear ratio required for the first and second reducers can be naturally implemented by selecting the output shaft.

As another example, the first reducer may include a first planetary gear assembly, and the second reducer may include a second planetary gear assembly.

In this case, the first planetary gear assembly may include a sun gear acting as an input, a carrier acting as an output, and a fixed ring gear.

And, the second planetary gear assembly may include a sun gear acting as an input, a ring gear acting as an output, and a fixed carrier.

In this way, the gear ratio required for the first and second reducers can be naturally implemented by selecting the output shaft.

A minimally invasive surgical robot having a belt-type remote center of motion mechanism according to the present disclosure may further include a base to which the input link is coupled; a surgical tool coupled to the output link; and an actuator installed at the base and providing a driving force for an insertion/exit motion of the surgical tool.

And, the power transmission member may include a third pulley coupled to the first pulley by a first axis and a fourth pulley positioned spaced apart from the third pulley; a first belt connecting the third pulley and the fourth pulley; a first scissor link having a first end coupled to the third pulley by a first axis and a second end coupled to the fourth pulley by a second axis; a fifth pulley coupled to the fourth pulley by the second axis and positioned spaced apart from the second pulley; a second belt connecting the second pulley and the fifth pulley; and a second scissor link having a first end coupled to the fourth pulley and the fifth pulley and a second end coupled to the second pulley by a third axis.

A length of the first scissor link may be equal to a length of the second scissor link.

Here, the length of the first scissor link may be a separation distance between the first axis and the second axis, and the second length of the second scissor link may be a separation distance between the second axis and the third axis.

A separation distance between the third pulley and the fourth pulley may be equal to a separation distance between the second pulley and the fifth pulley.

Here, the separation distance between the third pulley and the fourth pulley may be a separation distance between the first axis and the second axis, and the separation distance between the second pulley and the fifth pulley may be a separation distance between the second axis and the third axis.

To allow the second pulley to move along a longitudinal direction of the input link, the input link may be provided with a prismatic joint.

Alternatively, the input link may be formed as a lead screw to allow the second pulley to move along a longitudinal direction of the input link.

In the minimally invasive surgical robot, when the surgical tool coupled to an end of the input link is inserted, the second pulley moves along the longitudinal direction of the input link, and an angle between the first scissor link and the second scissor link can increase or decrease while the second pulley moves along a longitudinal direction of the input link.

And, when the pitch and roll motions of the surgical tool coupled to the end of the input link are performed, a position of the second pulley relative to the input link can be fixed, and the angle between the first scissor link and the second scissor link can be maintained constant.

The belt-type remote center of motion mechanism disclosed in the present disclosure and the minimally invasive surgical robot equipped with the same can be utilized in minimally invasive surgery on hard tissues (for example, bones, joints, etc.) that require great force.

In addition, the rigidity can be enhanced by reducing the rotational speed of the pulley by the reducer and then transmitting it to the link to reduce the tension transmitted to the belt.

And, the constraint conditions for remote center of motion operation can be maintained by adjusting the gear ratio of the reducer.

In addition, since not only the 2-DOF rotation (pan/tilt) around a remote center of motion (i.e., human body entry point) but also the insertion motion of the surgical tool can be driven by the actuator at the base of the robot, a mass and a size of the output link portion can be reduced.

In addition, when using multiple surgical robot arms, interference between output links can be reduced, and constraints for remote center of motion can be maintained even during the insertion/exit motion of the surgical tools.

Effects that could be achieved with the present disclosure are not limited to those that have been described hereinabove merely by way of example, and other effects and advantages of the present disclosure will be more clearly understood from the following description by a person skilled in the art to which the present disclosure pertains.

### BRIEF DESCRIPTION OF THE DRAWINGS

The accompanying drawings, which are included to provide a further understanding of the present disclosure and constitute a part of the detailed description, illustrate embodiments of the present disclosure and serve to explain technical features of the present disclosure together with the description.

FIG. 1 is a drawing explaining the 4-DOF motion of a surgical tool in a minimally invasive surgical robot.

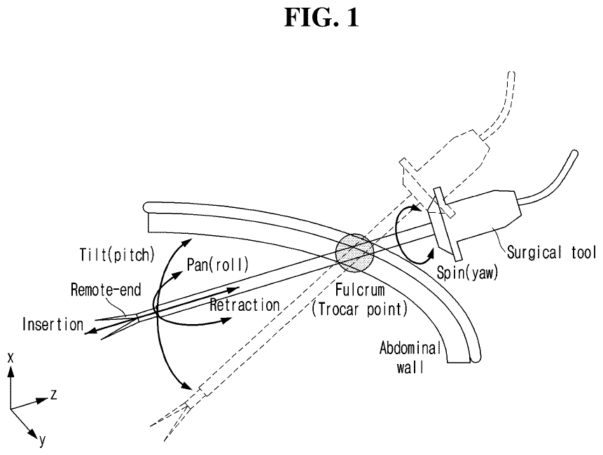

FIG. 2 is a drawing explaining the principle of a belt-type remote center of motion mechanism.

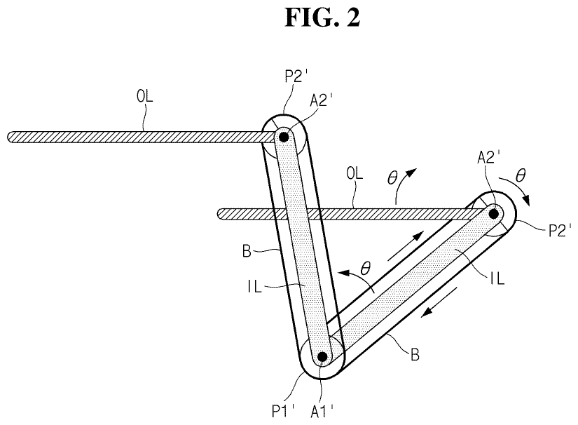

FIG. 3 is a drawing explaining the principle of displacement occurring in the output link of a surgical robot in a belt-type remote center of motion mechanism.

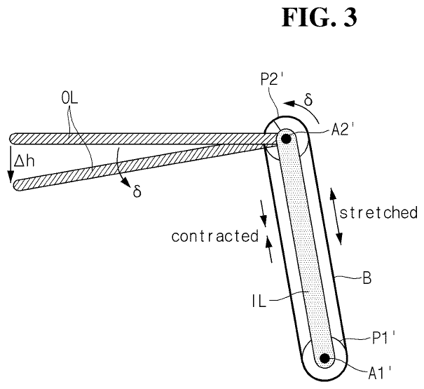

FIG. 4 is a drawing explaining the principle of a belt-type remote center of motion mechanism according to an embodiment of the present disclosure.

FIG. 5 is a drawing showing an example of a first reducer and a second reducer.

FIG. 6 is a drawing showing another example of a first reducer and a second reducer.

FIG. 7 is a perspective view of a minimally invasive surgical robot having a belt-type remote center of motion mechanism according to an embodiment of the present disclosure.

FIG. 8 is a front view of a minimally invasive surgical robot having a belt-type remote center of motion mechanism according to an embodiment of the present disclosure.

FIG. 9 is a left side view of a minimally invasive surgical robot having a belt-type remote center of motion mechanism according to an embodiment of the present disclosure.

FIG. 10 is a table showing the results of a rigidity evaluation experiment of a belt-type remote center of motion mechanism according to an embodiment of the present disclosure.

### DETAILED DESCRIPTION OF THE EMBODIMENTS

Reference will now be made in detail to embodiments of the present disclosure, examples of which are illustrated in the accompanying drawings. Wherever possible, the same reference numbers will be used throughout the drawings to refer to the same or like parts.

In general, a suffix such as “assembly” and “unit” may be used to refer to elements or components. Use of such a suffix herein is merely intended to facilitate description of the present disclosure, and the suffix itself is not intended to give any special meaning or function.

It will be noted that a detailed description of known arts will be omitted if it is determined that the detailed description of the known arts can obscure embodiments of the present disclosure.

The accompanying drawings are used to help easily understand various technical features and it should be understood that embodiments presented herein are not limited by the accompanying drawings. As such, the present disclosure should be understood to extend to any alterations, equivalents and substitutes in addition to those which are particularly set out in the accompanying drawings.

The terms including an ordinal number such as first, second, etc. may be used to describe various components, but the components are not limited by such terms. The terms are used only for the purpose of distinguishing one component from other components.

When any component is described as “being coupled to” or “being assembled with” other component, this should be understood to mean that another component may exist between them although any component may be directly coupled to or assembled with the other component.

On the other hand, when any component is described as “being directly coupled to” or “being assembled with” other component, this should be understood to mean that no component exists between them.

A singular expression can include a plural expression as long as it does not have an apparently different meaning in context.

In embodiments of the present disclosure, terms “include or comprise” or “have” should be understood to be intended to designate that illustrated features, numbers, steps, operations, components, parts or combinations thereof are present and not to preclude the existence of one or more other features, numbers, steps, operations, components, parts or combinations thereof, or the possibility of the addition thereof.

Hereinafter, the present disclosure will be described in detail with reference to the attached drawings. Regardless of the drawing symbols, identical or similar components will be given the same reference numbers and redundant descriptions thereof will be omitted.

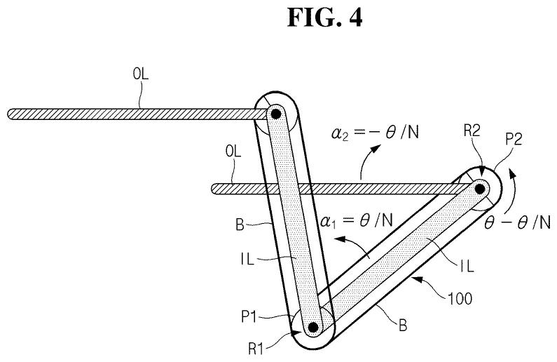

First, referring to FIGS. 4 to 6, a belt-type remote center of motion mechanism according to an embodiment of the present disclosure will be described.

FIG. 4 is a drawing explaining the principle of a belt-type remote center of motion mechanism according to an embodiment of the present disclosure.

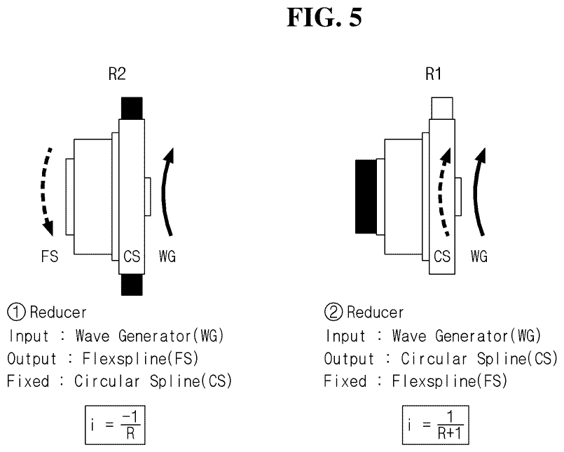

FIG. 5 is a drawing showing an example of a first reducer and a second reducer.

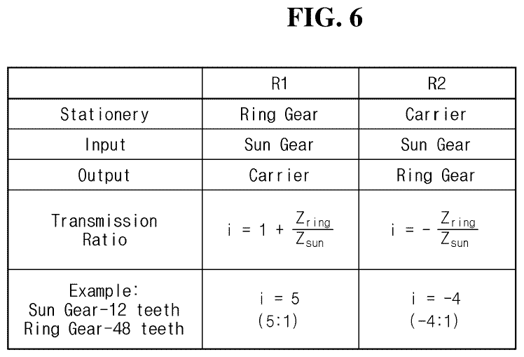

FIG. 6 is a drawing showing another example of a first reducer and a second reducer.

A belt-type remote center of motion mechanism according to an embodiment of the present disclosure includes an input link IL, an output link OL, a first pulley P1 and a second pulley P2, a first reducer R1 and a second reducer R2, and a power transmission member 100.

The first pulley P1 is located at a first end of the input link IL, and the second pulley P2 is located apart from the first pulley P1.

The output link OL has a first end connected to the second pulley P2 and receives a rotation of the second pulley P2.

The power transmission member 100 is provided to transmit a rotation of the first pulley P1 to the second pulley P2. In the belt-type remote center of motion mechanism of FIG. 4, the belt B connecting the first pulley P1 and the second pulley P2 acts as the power transmission member.

The first reducer R1 is located between the first pulley P1 and the input link IL, and reduces a rotational speed of the first pulley P1 and transmits it to the input link IL.

At this time, the first reducer R1 can reduce the rotational speed of the first pulley P1 with a gear ratio of N₁:1 and transmit it to the input link IL.

And the second reducer R2 is located between the second pulley P2 and the first end of the output link OL, and reduces a rotational speed of the second pulley P2 and transmits it to the output link OL.

At this time, the second reducer R2 can reduce the rotational speed of the second pulley P2 with a gear ratio of N₂:1 and transmit it to the output link OL.

Referring to FIG. 4, when the first pulley P1 rotates by θ, it can be seen that a rotation angle α1 of the input link IL becomes θ/N₁, and a rotation angle α2 of the output link OL becomes (θ−θ/N₁)/N₂.

Therefore, if N₂ becomes −(N₁−1), the rotation angle α2 of the output link OL becomes-θ/N₁, so the rotation angle α2 of the output link OL becomes −α1.

The rotation angle α2 of the above output link OL can be calculated by substituting a value of N2 into the above equation (θ−θ/N₁)/N₂). The value of N2 is −(N₁−1).

Therefore, by adjusting the reduction ratio of the first reducer R1 and the second reducer R2 so that N₂ becomes −(N₁−1), a remote center of motion operation can be implemented.

The first reducer R1 and the second reducer R2 may include a harmonic drive or a planetary gear assembly.

For example, the first reducer may include a first harmonic drive, and the second reducer may include a second harmonic drive.

Harmonic drive is a type of reducer that has the advantages of high precision, excellent repeatability, zero backlash, high torsional stiffness, high torque density, etc., and includes a wave generator, a circular spline, and a flex spline.

Also, a gear ratio of the harmonic drive can be adjusted depending on whether a circular spline or a flex spline is used as the output shaft.

In this embodiment, the first harmonic drive R1 may include a wave generator acting as an input, a circular spline acting as an output, and a fixed flex spline.

And the second harmonic drive R2 may include a wave generator acting as an input, a flex spline acting as an output, and a fixed circular spline.

In this way, the gear ratio required for the first and second reducers can be naturally implemented by selecting the output shaft.

As another example, the first reducer R1 may include a first planetary gear assembly, and the second reducer R2 may include a second planetary gear assembly.

The planetary gear assembly may include a sun gear, a ring gear, and a carrier that transmits power between the sun gear and the ring gear.

In the present disclosure, the first planetary gear assembly may include a sun gear acting as an input, a carrier acting as an output, and a fixed ring gear.

And, the second planetary gear assembly may include a sun gear acting as an input, a ring gear acting as an output, and a fixed carrier.

In this way, the gear ratio required for the first and second reducers can be naturally implemented by selecting the output shaft.

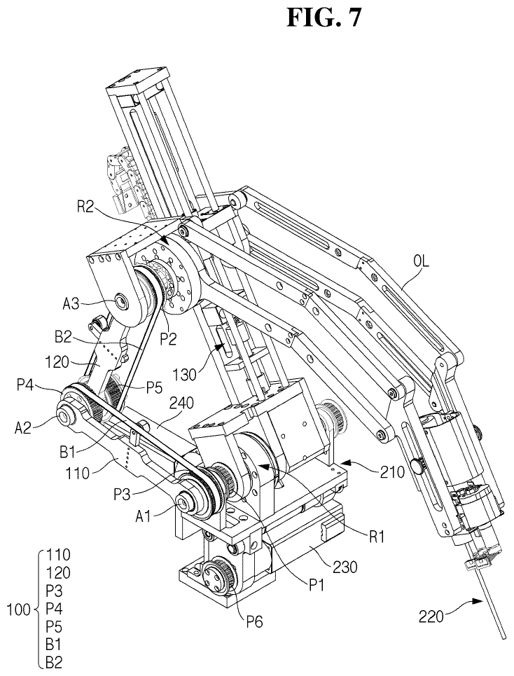

Hereinafter, with reference to FIGS. 7 to 10, the minimally invasive surgical robot equipped with a belt-type remote center of motion mechanism according to the present disclosure will be described.

FIG. 7 is a perspective view of a minimally invasive surgical robot having a belt-type remote center of motion mechanism according to an embodiment of the present disclosure.

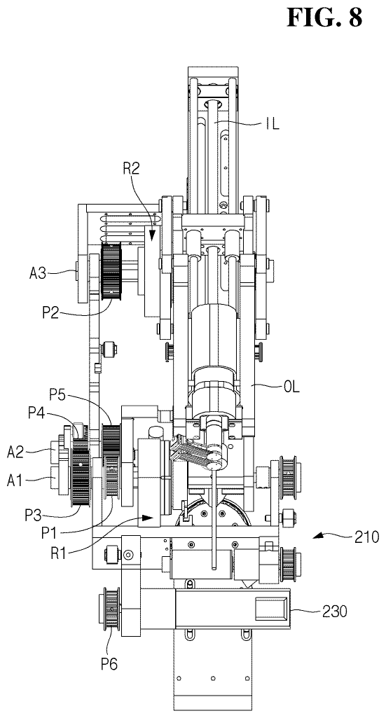

FIG. 8 is a front view of a minimally invasive surgical robot having a belt-type remote center of motion mechanism according to an embodiment of the present disclosure.

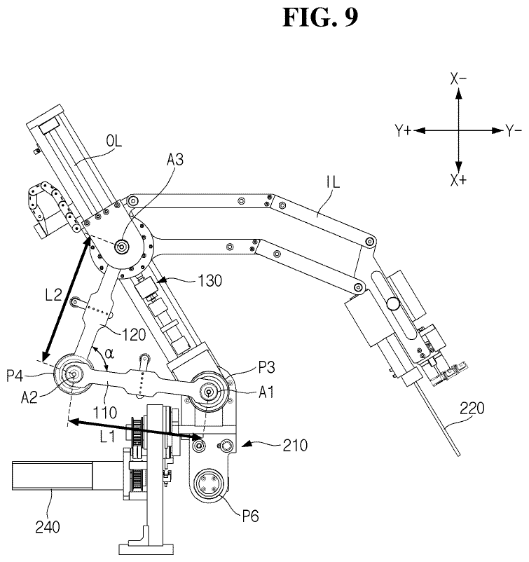

FIG. 9 is a left side view of a minimally invasive surgical robot having a belt-type remote center of motion mechanism according to an embodiment of the present disclosure.

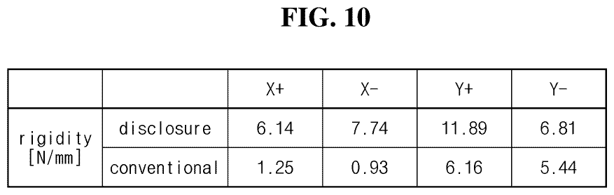

FIG. 10 is a table showing the results of a rigidity evaluation experiment of a belt-type remote center of motion mechanism according to an embodiment of the present disclosure.

The minimally invasive surgical robot of the present disclosure further includes a base 210 to which the input link IL is coupled, a surgical tool 220 coupled to the output link OL, and an actuator 230 installed in the base 210 and providing a driving force for an insertion/exit motion of the surgical tool 220.

And, in the minimally invasive surgical robot of the present disclosure, the power transmission member 100 may include a third pulley P3 coupled to the first pulley P1 by a first axis A1, a fourth pulley P4 positioned spaced apart from the third pulley P3, a first belt B1 connecting the third pulley P3 and the fourth pulley P4, a first scissor link 110 having a first end coupled to the third pulley P3 by the first axis A1 and a second end coupled to the fourth pulley P4 by the second axis A2, a fifth pulley P5 coupled to the fourth pulley P4 by the second axis A2 and positioned spaced apart from the second pulley P2, a second belt B2 connecting the second pulley P2 and the fifth pulley P5, and a second scissor link 120 having a first end coupled to the fourth pulley P4 and the fifth pulley P5 and a second end coupled to the second pulley P2 by a third axis A3.

Here, the first scissor link 110 and the second scissor link 120 can correspond to the input links of the remote center of motion mechanism illustrated in FIG. 4.

A first reducer R1 is provided between the first pulley P1 and the input link IL, and a second reducer R2 is provided between the second pulley P2 and the output link OL.

The second pulley P2 is installed so as to be able to move along a longitudinal direction of the input link IL.

To allow the second pulley P2 to move along the longitudinal direction of the input link IL, the input link IL may be provided with a prismatic joint 130.

Alternatively, the input link IL may be formed as a lead screw to allow the second pulley P2 to move along the longitudinal direction of the input link IL.

According to this configuration, while the second pulley P2 moves along the longitudinal direction of the input link IL, an angle α between the first scissor link 110 and the second scissor link 120 increases or decreases.

Specifically, while the second pulley P2 moves upward along the longitudinal direction of the input link IL, the angle α between the first scissor link 110 and the second scissor link 120 increases.

And while the second pulley P2 moves downward along the longitudinal direction of the input link IL, the angle α between the first scissor link 110 and the second scissor link 120 decreases.

A length L1 of the first scissor link 110 can be formed to be the same as a length L2 of the second scissor link 120.

Here, the length L1 of the first scissor link 110 is a separation distance between the first axis A1 and the second axis A2, and the second length L2 of the second scissor link 120 is a separation distance between the second axis A2 and the third axis A3.

The length L1 of the first scissor link 110 is formed to be the same as the length L2 of the second scissor link 120 to decoupling the insertion/exit motion and rotational motion of the surgical tool 220 coupled to the output link OL.

To explain this, when the prismatic joint 130 of the input link IL is extended by Δh due to the insertion/exit motion of the surgical tool 220, the first scissor link 110 rotates by Δθ₁ and the second scissor link 120 rotates by Δθ₂ in the opposite direction to the first scissor link 110.

Due to this, the second pulley P2 between the second scissor link 120 and the output link OL and the output link OL coupled to the pulley P2 rotate by 402-401.

Therefore, if the length L1 of the first scissor link 110 and the length L2 of the second scissor link 120 are formed to be the same, Δθ₂ and Δθ₁ become the same, so that the rotation of the first scissor link 110 and the second scissor link 120 due to the insertion/exit motion of the surgical tool 220 is canceled out, and rotation of the output link OL does not occur.

The fact that the length L1 of the first scissor link 110 is formed to be the same as the length L2 of the second scissor link 120 may mean, in another sense, that a separation distance between the first pulley P3 and the fourth pulley P4 is the same as a separation distance between the fifth pulley P5 and the second pulley P2.

Here, the separation distance between the third pulley P3 and the fourth pulley P4 is the separation distance between the first axis A1 and the second axis A2, and the separation distance between the fifth pulley P5 and the second pulley P2 is the separation distance between the second axis A2 and the third axis A3.

When the sizes of the second to fifth pulleys P2, P3, P4 and P5 are the same, a length of the first belt B1 connecting the third pulley P3 and the fourth pulley P4 may be the same as a length of the second belt B2 connecting the fifth pulley P5 and the second pulley P2.

According to the belt-type remote center of motion mechanism of this embodiment, when the surgical tool 220 coupled to the end of the output link OL undergoes an insertion/exit motion, the angle α between the first scissor link 110 and the second scissor link 120 can increase or decrease.

And, when the pitch and roll motions of the surgical tool 220 coupled to the end of the output link OL are performed, the angle α between the first scissor link 110 and the second scissor link 120 can be maintained constant without changing.

In the minimally invasive surgical robot of the present disclosure, a sixth pulley P6 is coupled to a shaft of the actuator 230, and the sixth pulley P6 is connected to the first pulley P1 that is axially coupled to the third pulley P3 by a first shaft A1 by a belt.

Therefore, when the actuator 230 is driven to rotate the sixth pulley P6, the sixth pulley P6 rotates, and accordingly, the input link IL rotates, causing an insertion/exit motion of the surgical tool 140.

Referring to FIGS. 9 and 10, it can be seen that the minimally invasive surgical robot of the present disclosure has greatly increased stiffness in the X-axis direction and Y-axis direction compared to conventional minimally invasive surgical robots.

In FIG. 10, a conventional minimally invasive surgical robot refers to a robot having a belt-type remote center of motion mechanism that does not have the first reducer R1 and the second reducer R2.

Therefore, in the minimally invasive surgical robot equipped with the belt-type remote center of motion mechanism of the present disclosure, since deceleration is performed by the first reducer R1 and the second reducer R2 during the driving process of the surgical tool 230, a torque applied to the link is reduced by 1/(N−1) times, and accordingly, the deformation of the belt is reduced by 1/(N−1) times, so that the rigidity of the robot can be effectively increased.

The minimally invasive surgical robot according to the present disclosure may also be equipped with an actuator 240 for pan (roll) motion at the base 210.

What is claimed is:

1. A belt-type remote center of motion mechanism, comprising:
an input link;
a first pulley positioned toward a first end side of the input link and a second pulley positioned spaced apart from the first pulley;
a power transmission member including a belt transmitting a rotation of the first pulley to the second pulley;
a first reducer located between the first pulley and the first end side of the input link and configured to reduce a rotational speed of the first pulley and transmit it to the input link;
an output link having a first end coupled to the second pulley and receiving the rotation of the second pulley; and
a second reducer positioned between the second pulley and the first end of the output link and configured to reduce a rotational speed of the second pulley and transmit it to the output link.

2. The belt-type remote center of motion mechanism of claim 1,
wherein the first reducer reduces the rotational speed of the first pulley with a gear ratio of N₁:1 and transmit it to the input link,
wherein the second reducer reduces the rotational speed of the second pulley with a gear ratio of N₂:1 and transmit it to the output link, and
wherein N₂ is −(N₁−1).

3. The belt-type remote center of motion mechanism of claim 1,
when the rotation angle of the first pulley is θ, a rotation angle of the first link is θ/N₁, and a rotation angle of the second link is −θ/N₁.

4. The belt-type remote center of motion mechanism of claim 1,
wherein the first reducer and the second reducer include a harmonic drive or a planetary gear assembly.

5. The belt-type remote center of motion mechanism of claim 4,
wherein the first reducer includes a first harmonic drive, and the second reducer includes a second harmonic drive.

6. The belt-type remote center of motion mechanism of claim 5,
wherein the first harmonic drive includes a wave generator acting as an input, a circular spline acting as an output, and a fixed flex spline.

7. The belt-type remote center of motion mechanism of claim 6,
wherein the second harmonic drive includes a wave generator acting as an input, a flex spline acting as an output, and a fixed circular spline.

8. A minimally invasive surgical robot having the belt-type remote center of motion mechanism of claim 1, further comprising:
a base to which the input link is coupled;
a surgical tool coupled to the output link; and
an actuator installed at the base and providing a driving force for an insertion/exit motion of the surgical tool,
wherein the power transmission member includes:
a third pulley coupled to the first pulley by a first axis and a fourth pulley positioned spaced apart from the third pulley;
a first belt connecting the third pulley and the fourth pulley;
a first scissor link having a first end coupled to the third pulley by a first axis and a second end coupled to the fourth pulley by a second axis;
a fifth pulley coupled to the fourth pulley by the second axis and positioned spaced apart from the second pulley;
a second belt connecting the second pulley and the fifth pulley; and
a second scissor link having a first end coupled to the fourth pulley and the fifth pulley and a second end coupled to the second pulley by a third axis.

9. The minimally invasive surgical robot of claim 8,
wherein a length of the first scissor link is equal to a length of the second scissor link.

10. The minimally invasive surgical robot of claim 9,
wherein the length of the first scissor link is a separation distance between the first axis and the second axis, and the second length of the second scissor link is a separation distance between the second axis and the third axis.

11. The minimally invasive surgical robot of claim 8,
wherein a separation distance between the third pulley and the fourth pulley is equal to a separation distance between the second pulley and the fifth pulley.

12. The minimally invasive surgical robot of claim 11,
wherein the separation distance between the third pulley and the fourth pulley is a separation distance between the first axis and the second axis, and the separation distance between the second pulley and the fifth pulley is a separation distance between the second axis and the third axis.

13. The minimally invasive surgical robot of claim 8,
wherein the input link is provided with a prismatic joint to allow the second pulley to move along a longitudinal direction of the input link.

14. The minimally invasive surgical robot of claim 8,
wherein the input link is formed as a lead screw to allow the second pulley to move along a longitudinal direction of the input link.

15. The minimally invasive surgical robot of claim 8,
wherein when the surgical tool coupled to an end of the input link is inserted, the second pulley moves along the longitudinal direction of the input link, and an angle between the first scissor link and the second scissor link increase or decrease while the second pulley moves along a longitudinal direction of the input link.

16. The minimally invasive surgical robot of claim 15,
wherein when the pitch and roll motions of the surgical tool coupled to the end of the input link are performed, a position of the second pulley relative to the input link is fixed, and the angle between the first scissor link and the second scissor link is maintained constant.
